(19) United States
(12) Patent Application Publication
(10) Pub. No.: US 2025/0281031 A1
(43) Pub. Date: Sep. 11, 2025

(54) ENDOSCOPE

(71) Applicant: OLYMPUS MEDICAL SYSTEMS CORP., Tokyo (JP)

(72) Inventor: Hidenosuke HASE, Tokyo (JP)

(73) Assignee: OLYMPUS MEDICAL SYSTEMS CORP., Tokyo (JP)

(21) Appl. No.: 19/070,868

(22) Filed: Mar. 5, 2025

Related U.S. Application Data
(60) Provisional application No. 63/561,925, filed on Mar. 6, 2024.

(51) Int. Cl. A61B 1/005

(52) U.S. Cl. CPC A61B 1/0057

(57) ABSTRACT

An endoscope includes a bending tube extending along a longitudinal axis from a proximal end side to a distal end side, a wire that bends the bending tube, a first sheath through which the wire is passed, and a plate body having an annular shape and disposed in the proximal end side of the bending tube. The plate body includes: a first annular surface facing distally; a second annular surface facing proximally and intersecting the longitudinal axis; and a hole penetrating the first annular surface and the second annular surface and through which the wire is passed, and at least a portion of the first sheath is disposed proximally relative to the second annular surface and a distal end of at least the portion of the first sheath is configured to push a periphery of the hole on the second annular surface.

### RELATED APPLICATION DATA

This application is based on and claims priority under 35 U.S.C. § 119 to U.S. Provisional Application No. 63/561,925, filed on Mar. 6, 2024, the entire contents of which are incorporated herein by reference.

### BACKGROUND

### 1. Field

The present disclosure relates to an endoscope including a bending tube that is bent by pulling or loosening a wire.

### 2. Related Art

Endoscopes including a bending tube (also referred to as a bending portion) have been proposed in the past. The bending tube changes the direction of a distal end portion by a bending wire being pulled or loosened, thereby changing the direction of observation with the endoscope. The bending wire is passed through from an operation portion of the endoscope to the bending tube.

In a flexible tube, the bending wire is passed through a sheath that guides the wire. In this case, a configuration may be adopted to reduce the friction induced between the sheath and the wire.

For example, if watertightness in the insertion portion can be ensured, lubricants such as silicone oil and carbon powder may be used to reduce the friction.

If watertightness in the insertion portion cannot be ensured, an inner sheath may be provided that has less friction with the wire than the sheath. At this time, the wire is passed through the sheath while being in a state of being passed through the inner sheath.

For example, Japanese Patent No. 6028136 describes using, as a sheath through which a bending wire is to be passed, a coil sheath formed by winding a core wire in a coil shape. Specifically, the bending wire is passed through an inner coil sheath, and is further passed through an outer coil sheath. A distal end portion of the outer coil sheath is fixed to an inner surface of the flexible tube on a distal end side, and a proximal end surface of the outer coil sheath is fixed to a fixing plate in an operation portion.

### SUMMARY

An endoscope according to an aspect of the present disclosure includes a bending tube extending along a longitudinal axis from a proximal end side to a distal end side, a wire configured to bend the bending tube, a first sheath through which the wire is passed, and a plate body having an annular shape and disposed in the proximal end side of the bending tube. The plate body includes: a first annular surface facing distally; a second annular surface facing proximally and intersecting the longitudinal axis; and a hole penetrating the first annular surface and the second annular surface and through which the wire is passed, and at least a portion of the first sheath is disposed proximally relative to the second annular surface, and a distal end of at least the portion of the first sheath is configured to push a periphery of the hole on the second annular surface.

An endoscope according to an aspect of the present disclosure includes a bending tube extending along a longitudinal axis from a proximal end side to a distal end side, a wire configured to bend the bending tube, a first sheath through which the wire is passed, and a plate body having an annular shape and disposed in the proximal end side of the bending tube. The plate body includes: a first annular surface facing distally; a second annular surface facing proximally and intersecting the longitudinal axis; and a hole penetrating the first annular surface and the second annular surface and through which the wire is passed, and at least a portion of the first sheath is disposed proximally relative to the second annular surface, and a distal end of at least the portion of the first sheath is configured to produce a frictional force in a direction intersecting the longitudinal axis between the distal end of at least the portion and a periphery of the hole on the second annular surface.

An endoscope according to an aspect of the present disclosure includes a bending tube extending along a longitudinal axis from a proximal end side to a distal end side, a wire configured to bend the bending tube, a first sheath, a second sheath passed through the first sheath and through which the wire is passed, and a plate body having an annular shape and disposed in the proximal end side of the bending tube. The plate body includes: a first annular surface facing distally; a second annular surface facing proximally; and a hole penetrating the first annular surface and the second annular surface and through which the wire is passed, at least a portion of the first sheath is disposed proximally relative to the second annular surface, and a distal end of at least the portion of the first sheath is configured to push a periphery of the hole on the second annular surface, and the wire and the second sheath are passed through the hole and a distal end of the second sheath is disposed distally relative to the hole.

### BRIEF DESCRIPTION OF THE DRAWINGS

FIG. 1 is a perspective view showing a configuration of an endoscope of a first embodiment of the present disclosure with some portions omitted.

FIG. 2 is a cross-sectional view showing a configuration of a bending wire at a connection part between a bending tube and a flexible tube in the first embodiment.

FIG. 3 is a partial perspective view showing a configuration of the bending wire, an inner sheath, a sheath, and a plate body in the first embodiment.

FIG. 4 is a cross-sectional view of the bending tube near the plate body, taken orthogonal to a longitudinal axis, in the first embodiment.

FIG. 5 is a cross-sectional view of a modification of the plate body in the first embodiment, when cut in a plane parallel to the longitudinal axis.

FIG. 6 is a cross-sectional view showing, from an oblique direction, a configuration of a second plate body disposed on a distal end side of the bending tube in the first embodiment.

FIG. 7 is a cross-sectional view showing a wire that is passed through four holes in the second plate body in the first embodiment.

FIG. 8 is a cross-sectional view showing two wires that are respectively passed through two holes in the second plate body in the first embodiment.

FIG. 9 is a perspective view showing a fixing body disposed in an operation portion in the first embodiment.

FIG. 10 is a cross-sectional view showing a portion of a structure of the fixing body in the first embodiment.

FIG. 11 is a view showing how a position to cut the sheath is set according to an actual length of a flexible tube body in the first embodiment.

FIG. 12 is a side view showing an operation mechanism disposed in the operation portion in the first embodiment.

FIG. 13 is a partial perspective view showing the operation mechanism of the first embodiment from a direction of an arrow A1 in FIG. 12.

FIG. 14 is a view for illustrating a process of fixing the bending wire to the operation mechanism in the first embodiment.

FIG. 15 is a view showing that the sheath is held in a state of pressing the plate body and the fixing body in the first embodiment.

FIG. 16 is a cross-sectional view showing a relationship between the sheath and the wire when the inner sheath is not provided in the first embodiment.

FIG. 17 is a view showing a configuration example of the sheath in a second embodiment of the present disclosure.

FIG. 18 is a view showing a first example of a swing angle of a wire in a third embodiment of the present disclosure.

FIG. 19 is a view showing a second example of the swing angle of the wire in the third embodiment.

FIG. 20 is a view showing a configuration example of a sheath in a fourth embodiment of the present disclosure.

FIG. 21 is diagram showing a configuration example of a part of a second annular surface of a plate body, where a distal end of a sheath abuts, in a fifth embodiment of the present disclosure.

### DETAILED DESCRIPTION

In general, a sheath through which a bending wire is passed is subjected to a pulling force load when the bending wire is pulled to bend a bending tube. For this reason, the sheath is firmly fixed to a distal end side of a flexible tube or a proximal end side of the bending tube by welding or the like, for example.

However, welding or the like is not suitable for manufacturing inexpensive endoscopes because skilled workers must use jigs to perform the work.

According to embodiments described below, an endoscope can be provided including a sheath through which a bending wire is passed, suitable for manufacturing inexpensive endoscopes.

The following describes embodiments of the present disclosure with reference to the drawings. However, the present disclosure is not limited by the embodiments described below.

Note that, in the description of the drawings, the same or corresponding elements are attached with the same symbol as appropriate. It should be noted that the drawings are schematic, and the relationship of lengths of each element, the ratio of lengths of each element, the quantity of each element, etc. within a single drawing may differ from reality for the sake of brevity of explanation. Furthermore, even among a plurality of drawings, they may contain portions that differ from each other in terms of length relationships, ratios, quantities, etc.

### FIRST EMBODIMENT

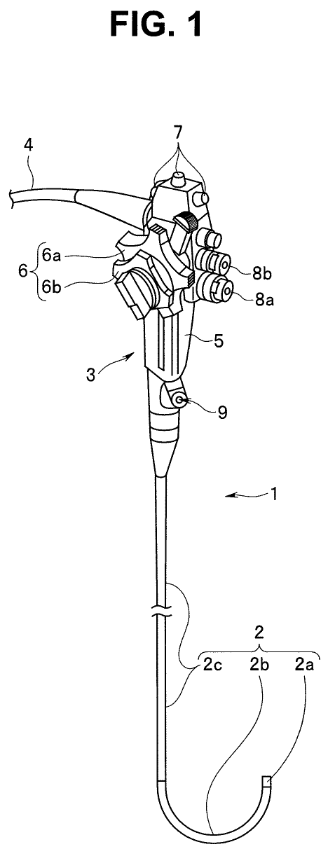

FIGS. 1 to 16 show a first embodiment of the present disclosure. FIG. 1 is a perspective view showing a configuration of an endoscope 1 of a first embodiment with some portions omitted.

The endoscope 1 is an insertion instrument including a section to be inserted into a subject. The subject can be a living body such as a human or an animal, or a non-living body such as a machine or a building.

The endoscope 1 includes an insertion portion 2, an operation portion 3, and a universal cable 4.

The insertion portion 2 is a section configured to be inserted into the subject. The insertion portion 2 includes a distal end portion 2a, a bending tube 2b, and a flexible tube 2c, in order from the distal end side to the proximal end side.

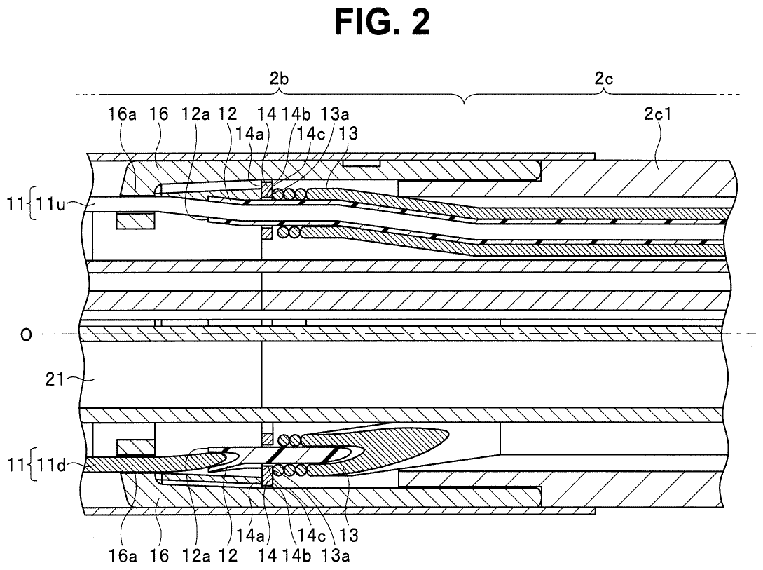

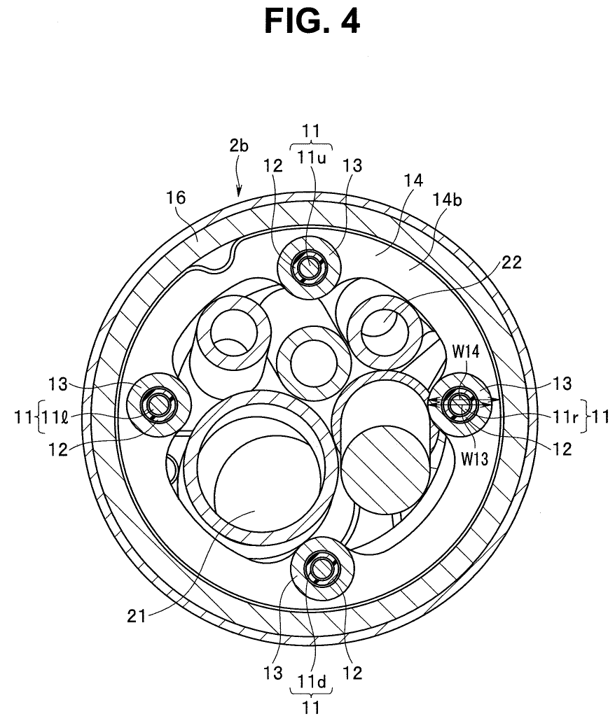

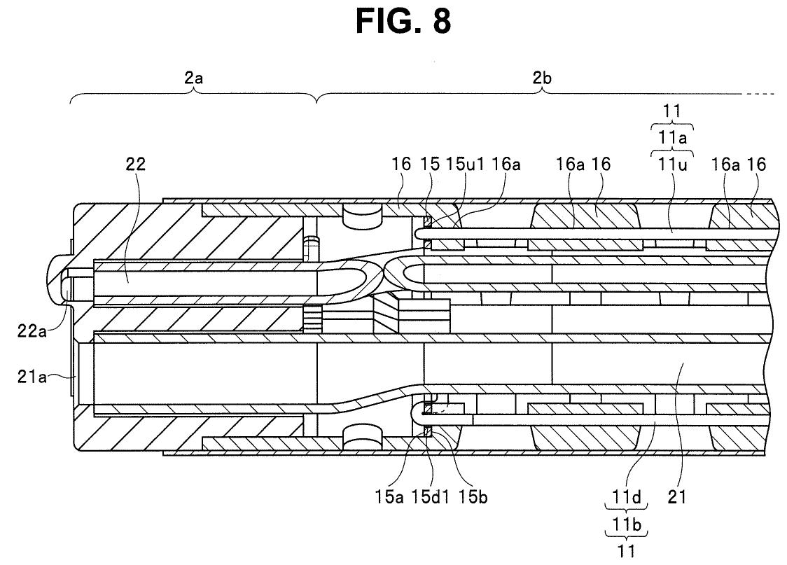

In the distal end portion 2a, an image pickup device, an image pickup optical system, an illumination optical system, a nozzle 22a (see FIG. 8), a channel distal-end-side opening 21a (see FIG. 8), etc., are disposed. The illumination optical system irradiates the subject with illumination light. The image pickup optical system forms an optical image of the subject. The image pickup device photoelectrically converts the optical image formed by the image pickup optical system, to generate an image pickup signal. The nozzle 22a discharges gas and liquid fed via a gas/liquid feeding channel 22 (see FIGS. 4, 6, 8, etc.) to an observation window at a distal end of the image pickup optical system. The channel distal-end-side opening 21a is an opening on the distal end side of a treatment instrument channel 21 (see FIGS. 2, 4, 6, etc.).

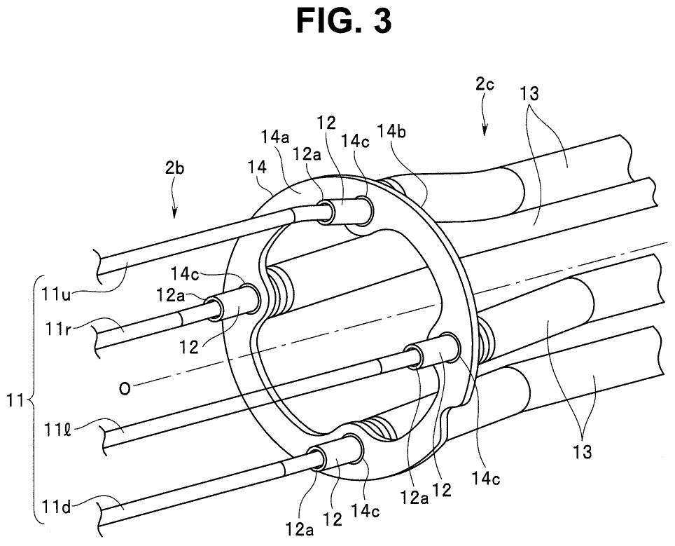

The bending tube 2b is a section that is bendable, for example, in four directions (up, down, left, and right). The bending tube 2b is also referred to as a bending portion. The bending tube 2b is provided along a longitudinal axis O (see FIGS. 2, 3, etc.) that extends from the proximal end side to the distal end side of the endoscope 1.

The flexible tube 2c is a tube portion having flexibility, is also referred to as a flexible tube portion. The flexible tube 2c is provided along the longitudinal axis O, on the proximal end side of the bending tube 2b. Note that here, an example is given in which the endoscope 1 is a flexible endoscope including the flexible tube 2c. However, the endoscope 1 can also be a rigid endoscope in a form where the part corresponding to the flexible tube 2c is rigid.

The operation portion 3 is provided on the proximal end side of the flexible tube 2c of the insertion portion 2. The operation portion 3 is a section for the user to operate the endoscope 1. The operation portion 3 includes a grasping portion 5, a bending operation knob 6, various operation switches 7, a gas/liquid feeding button 8a, a suction button 8b, and a treatment-instrument insertion opening 9.

The grasping portion 5 is a section where the user grasps the endoscope 1 with the palm.

The bending operation knob 6 is an operation device for operating the bending of the bending tube 2b. The bending operation knob 6 is operated, for example, using the thumb of the hand grasping the grasping portion 5. When the bending operation knob 6 is operated, the bending wire 11 (see FIGS. 2, 3, etc.) is pulled and the bending tube 2b is bent.

The bending operation knob 6 includes an up-down bending operation knob 6a and a left-right bending operation knob 6b. The up-down bending operation knob 6a is operated to bend the bending tube 2b in the up and down directions. The left-right bending operation knob 6b is operated to bend the bending tube 2b in the left and right directions.

When the bending tube 2b is bent, the direction of the distal end portion 2a changes. This changes the image pickup direction by the image pickup device and the image pickup optical system and the illumination direction of the illumination light by the illumination optical system. The bending tube 2b is bent also to improve insertability of the insertion portion 2 in the subject.

The operation switches 7 include a button switch relating to image pickup. Specific examples of the operation switches 7 are button switches such as a freeze button to pause the monitor screen and a release button to pick up a still image.

The gas/liquid feeding button 8a is a button for an operation of feeding gas and liquid to the observation window of the distal end portion 2a. The liquid feeding cleans the observation window, and the gas feeding blows away the liquid after the cleaning. The gas feeding and liquid feeding are performed via the gas/liquid feeding channel 22.

The suction button 8b is a button for performing an operation of suctioning the inside of the subject from the distal end portion 2a. The suction from inside the subject is performed, for example, via the treatment instrument channel 21, which also serves as a suction channel. When the suction operation is performed, for example, liquid or mucous membrane is suctioned from inside the subject.

The treatment-instrument insertion opening 9 is an opening on the proximal end side of the treatment instrument channel 21. A treatment instrument, such as a forceps, is inserted from the treatment-instrument insertion opening 9 into the treatment instrument channel 21. A distal end portion of the treatment instrument is guided from the treatment instrument channel 21 to the channel distal-end-side opening 21a, and protrudes into the subject. The distal end portion of the protruded treatment instrument is used to perform various treatments on the subject.

The universal cable 4 is extended from, for example, a side surface on the proximal end side of the operation portion 3. A connector is provided at an extended end of the universal cable 4. The connector connects the endoscope 1 to an endoscope processor (video processor), a light source apparatus, a suction pump, a liquid feeding tank, etc.

FIG. 2 is a cross-sectional view showing a configuration of the bending wire 11 at a connection part between the bending tube 2b and the flexible tube 2c in the first embodiment. FIG. 3 is a partial perspective view showing a configuration of the bending wire 11, an inner sheath 12, a sheath 13, and a plate body 14 in the first embodiment. FIG. 4 is a cross-sectional view of the bending tube 2b near the plate body 14, taken orthogonal to the longitudinal axis O, in the first embodiment. FIG. 4 is a view of a cross section viewed toward a distal end direction along the longitudinal axis O.

The wire 11 is configured to be pulled or loosened to bend the bending tube 2b.

In the case where the bending tube 2b is bendable in four directions (up, down, left, and right), the bending wire 11 includes an up-bending wire 11u, a down-bending wire 11d, a left-bending wire 11l, and a right-bending wire 11r.

The up-bending wire 11u is pulled to thereby bend the bending tube 2b in the up direction. The down-bending wire 11d is pulled to thereby bend the bending tube 2b in the down direction. The left-bending wire 11l is pulled to thereby bend the bending tube 2b in the left direction. The right-bending wire 11r is pulled to thereby bend the bending tube 2b in the right direction.

The bending wires 11u, 11d, 11r, and 11l in the respective directions are respectively passed through four inner sheaths 12 (second sheaths) so as to be slidable. Furthermore, the four inner sheaths 12, through which the bending wires 11u, 11d, 11r, and 11l are passed, are respectively passed through four sheaths 13 (first sheaths). Thus, the wires 11 are passed through the sheaths 13 via the inner sheaths 12.

The inner sheath 12 may be formed of a thermoplastic elastomer, which is a resin, and may be formed of, for example, PTFE (polytetrafluoroethylene) or PP (polypropylene) based resin, etc.

The sheath 13 is configured as, for example, a coil sheath having elasticity, and, in a compressed state in the direction of the longitudinal axis O relative to the natural length, is mounted in the endoscope 1. Note that the sheath 13 configured as a coil sheath has a coil shape over the entire length, but in the drawings, the illustration is simplified as appropriate, and parts other than end portions (such as near the plate body 14) are shown in a cylindrical shape. However, only a part of the entire length of the sheath 13 may be formed in a coil shape.

Here, a friction coefficient (first friction coefficient) between the inner sheath 12 and the wire 11 is less than a friction coefficient (second friction coefficient) between the sheath 13 and the wire 11. Note that by providing the inner sheath 12, which reduces friction, the use of a lubricant such as silicon oil or carbon powder becomes unnecessary, and the need to ensure watertightness in the insertion portion 2 is eliminated, resulting in a configuration suitable for manufacturing inexpensive endoscopes.

The plate body 14 having an annular shape is disposed in the proximal end side of the bending tube 2b. The plate body 14 is a ring member configured as a separate body from the bending tube 2b.

As shown in FIGS. 2 to 4, the plate body 14 includes a first annular surface 14a facing distally, a second annular surface 14b facing proximally, and a hole 14c penetrating the first annular surface 14a and the second annular surface 14b. The second annular surface 14b intersects the longitudinal axis O. The second annular surface 14b is orthogonal to the longitudinal axis O.

At least a portion of the sheath 13 is disposed on the proximal end side of the plate body 14. At least the portion of the sheath 13 is disposed proximally relative to the second annular surface 13b. In the examples shown in FIGS. 2 and 3, the entirety of the sheath 13 is disposed on the proximal end side of the plate body 14. The sheath 13 is mounted in the endoscope 1, in a compressed state, as mentioned above. For this reason, a distal end 13a of the sheath 13 pushes a periphery part of the hole 14c, the periphery part being of the second annular surface 14b, by a restoring force produced by the sheath 13 being compressed.

In other words, the distal end 13a of the sheath 13 exerts a normal force against the periphery part of the hole 14c, the periphery part being of the second annular surface 14b. The distal end 13a of the sheath 13 then produces a frictional force in a direction intersecting the longitudinal axis O between the distal end 13a of the sheath 13 and the periphery part of the hole 14c, the periphery part being of the second annular surface 14b.

At this time, the distal end 13a of the sheath 13 stably butts against the second annular surface 14b, since the second annular surface 14b is orthogonal to the longitudinal axis O. Therefore, the plate body 14 does not require a configuration, such as a long hole, through which the sheath 13 is passed, for causing the distal end 13a to stably butt against the second annular surface 14b, thus enabling to reduce the thickness of the plate body 14 in the longitudinal axis O direction. Note that, if necessary, the second annular surface 14b can be configured to obliquely intersect the longitudinal axis O.

The plate body 14 is sandwiched between the distal end 13a of the sheath 13 and the proximal end side of the bending tube 2b (for example, a guide member 16 disposed on the proximal-most end side of a plurality of guide members 16 disposed in the bending tube 2b).

The plate body 14 is neither welded nor bonded to the bending tube 2b. The plate body 14 abuts the proximal end side of the bending tube 2b (butted against the proximal end side of the bending tube 2b), and is fixed to the proximal end side of the bending tube 2b by the pressing force received from the sheath 13. Thus, the plate body 14 functions as a sheath stopper plate that locks the distal end 13a of the sheath 13.

The wire 11 in a state of being passed through the inner sheath 12 is passed through the hole 14c. Thus, a distal end 12a of the inner sheath 12 is disposed on the distal end side with respect to the hole 14c.

A part of the inner sheath 12 that is passed through the hole 14c of the plate body 14 is bonded to the hole 14c. The inner sheath 12 and the plate body 14 are unitized by bonding. The plate body 14 functions as a member to fix the distal end side of the inner sheath 12. Note that the proximal end side of the inner sheath 12 is disposed and is not fixed in the operation portion 3.

Note that here, a configuration is adopted where the distal end side of the inner sheath 12 is bonded and fixed to the hole 14c of the plate body 14 disposed in the bending tube 2b, and the proximal end side of the inner sheath 12 is left free. In this case, there is an advantage that the manufacturing process for fixing the inner sheath 12 is made easier.

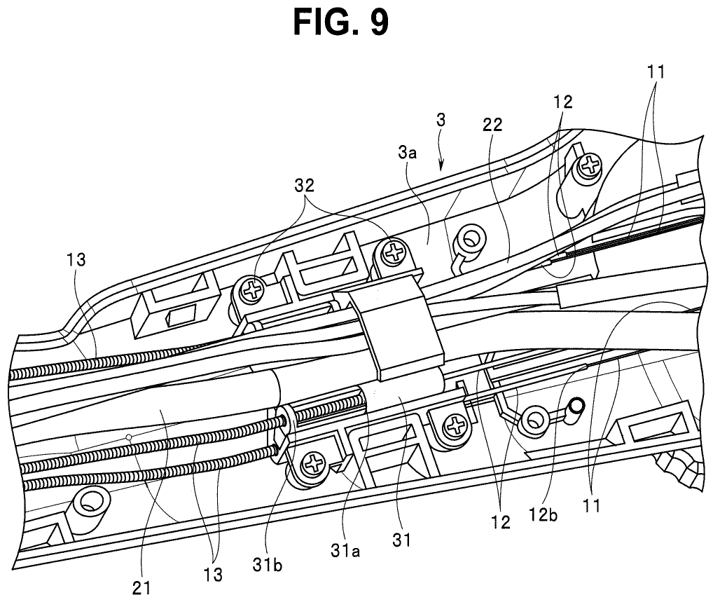

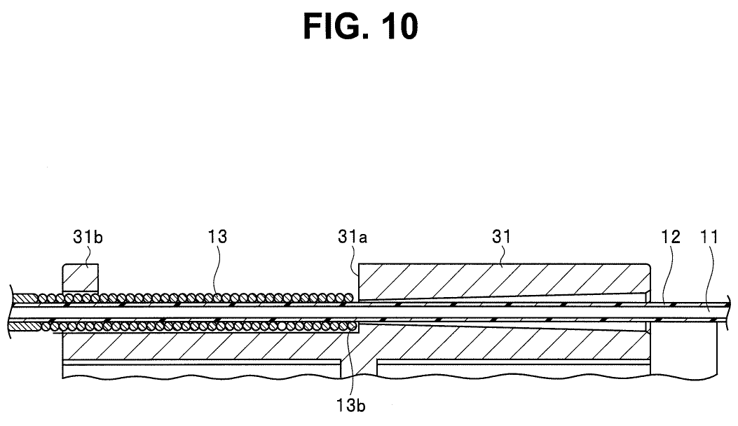

However, the inner sheath 12 is not limited to this configuration. For example, a configuration can be adopted where the proximal end side of the inner sheath 12 is fixed to a member disposed in the operation portion 3 (such as, for example, a fixing body 31 shown in FIGS. 9 and 10 described later), and the distal end side of the inner sheath 12 is left free. Furthermore, if there is no hindrance in use, the inner sheath 12 can be left free without being fixed.

The bending tube 2b includes a plurality of guide members 16 (see FIG. 8, etc.) that hold (guide) the wire 11. The plurality of guide members 16 each serve as a bending piece, for example. The guide member 16 disposed on a proximal-most end side of the bending tube 2b is connected to a flexible tube body 2c1 of the flexible tube 2c, as shown in FIG. 2.

The hole 14c is provided at a position (first radial position) different from a position (second radial position) where the guide member 16 holds the wire 11, in a plane orthogonal to the longitudinal axis O.

The guide member 16 include, for example, a hole 16a that functions as a guide hole for the wire 11. The wire 11 is passed through the hole 16a in a movable manner in a direction parallel to the longitudinal axis O. The position of the hole 16a in a plane orthogonal to the longitudinal axis O is a position where the guide member 16 holds the wire 11.

By differentiating the position of the hole 14c in the plate body 14 and the position of the hole 16a in the guide member 16 (here, especially positions in a radial direction with the longitudinal axis O at the center) in the plane orthogonal to the longitudinal axis O, a swing angle is produced in the wire 11 entering from the flexible tube 2c to the bending tube 2b.

This enables to give a swing angle to the wire 11 without providing an oblique long hole in the plate body 14. Therefore, the thickness of the plate body 14 in the longitudinal axis O direction can be reduced. This shortens the length of a non-flexible part of the insertion portion 2 in the longitudinal axis O direction, thereby improving the insertability of the insertion portion 2.

In the example shown in FIG. 2, the hole 14c is provided at a position closer to the longitudinal axis O than the hole 16a where the guide member 16 holds the wire 11, in the plane orthogonal to the longitudinal axis O. In this case, the hole 16a is located radially outward with respect to the hole 14c, with the longitudinal axis O as the center.

The wire 11 then produces a swing angle to a radially outward side when entering the bending tube 2b from the flexible tube 2c. It is thus enabled to more reliably avoid contact between the wire 11 and other internal components in the bending tube 2b, including when the bending tube 2b is bent.

As shown in FIG. 4, a width W14 of a periphery part of the hole 14c of the second annular surface 14b is greater than or equal to a width W13 of the distal end 13a of the sheath 13. In this case, the entire surface of the distal end 13a of the sheath 13 abuts the second annular surface 14b. As a result, the distal end 13a of the sheath 13 butts against the second annular surface 14b in stable manner.

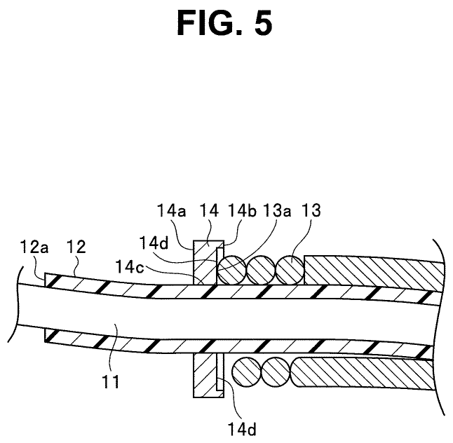

FIG. 5 shows a cross-sectional view of a modification of the plate body 14 in the first embodiment, when cut in a plane parallel to the longitudinal axis O.

The plate body 14 shown in the modification in FIG. 5 includes a groove 14d in the periphery part of the hole 14c in the second annular surface 14b on the proximal end side. The groove 14d is configured to be butted against by the distal end 13a of the sheath 13. By providing the groove 14d, it is enabled to suppress shifting of the position against where the distal end 13a butts, in a direction orthogonal to the longitudinal axis O, thereby stabilizing the butting.

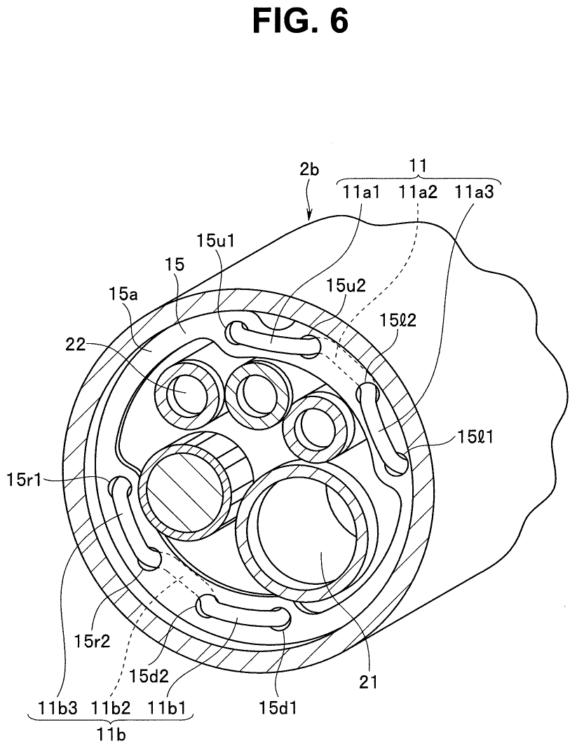

FIG. 6 is a cross-sectional view showing, from an oblique direction, a configuration of a second plate body 15 disposed in the distal end side of the bending tube 2b in the first embodiment.

The endoscope 1 includes the second plate body 15 disposed in the distal end side of the bending tube 2b. The second plate body 15 is disposed to intersect the longitudinal axis O. The second plate body 15 includes a distal-end-side surface 15a and a proximal-end-side surface 15b. The second plate body 15 is disposed in the distal end side of the bending tube 2b such that the distal-end-side surface 15a and the proximal-end-side surface 15b are orthogonal to the longitudinal axis O, for example.

Incidentally, in the present embodiment, the up-bending wire 11u and the left-bending wire 11l are coupled together on the distal end side, and configured of a single wire 11a. To put it another way, the single wire 11a has a portion that functions as the up-bending wire 11u and another portion that functions as the left-bending wire 11l.

Furthermore, the down-bending wire 11d and the right-bending wire 11r are coupled together on the distal end side, and configured of a single wire 11b. To put it another way, the single wire 11b has a portion that functions as the down-bending wire 11d and another portion that functions as the right-bending wire 11r.

Thus, the bending wire 11 is configured to perform up-down and left-right bending by a total of the two wires 11a and 11b.

In such a configuration, the second plate body 15 further includes a plurality of holes penetrating the distal-end-side surface 15a and the proximal-end-side surface 15b.

Specifically, the second plate body 15 includes, as the plurality of holes, four holes (but not limited to four, but may be an even number of two, or six, or more) for the wire 11a to be passed through. The four holes include a first hole 15u1, a second hole 15u2, a third hole 15l2, and a fourth hole 15l1, in order around the longitudinal axis O (for example, clockwise in FIG. 6).

Concerning the wire 11a, from the proximal end side of the second plate body 15, the wire 11u is passed, in order, through the first hole 15u1, is further passed through the second hole 15u2 from a distal end side of the second plate body 15, through the third hole 15l2 from the proximal end side of the second plate body 15, and through the fourth hole 15l1 from the distal end side of the second plate body 15, and the wire 11l is extended to the proximal end side of the second plate body 15.

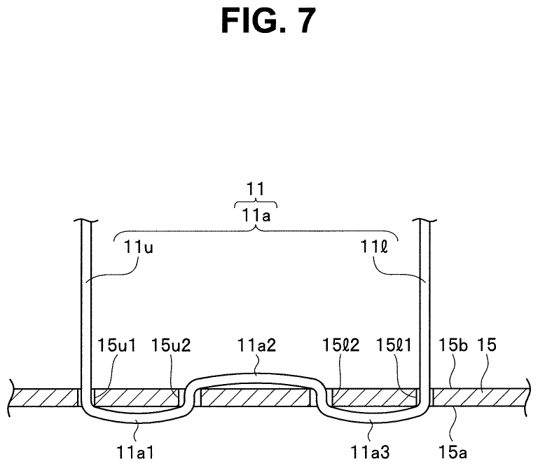

FIG. 7 is a cross-sectional view showing the wire 11a that is passed through the four holes 15u1, 15u2, 15l2, and 15l1 in the second plate body 15 in the first embodiment. FIG. 8 is a cross-sectional view showing the two wires 11a and 11b that are respectively passed through the two holes 15u1 and 15d1 in the second plate body 15 in the first embodiment.

The wire 11a has the wire 11u passed through the first hole 15u1 from the proximal end side of the insertion portion 2, and is extended as a first part 11a1 of the wire 11a to the distal-end-side surface 15a. The first part 11a1 of the wire 11a is disposed on the distal-end-side surface 15a in a circumferential direction, and is passed through the second hole 15u2.

The wire 11a passed through the second hole 15u2 is extended to the proximal-end-side surface 15b as a second part 11a2. The second part 11a2 is disposed on the proximal-end-side surface 15b in the circumferential direction, and is passed through the third hole 15l2.

The wire 11a passed through the third hole 15l2 is extended to the distal-end-side surface 15a as a third part 11a3. The third part 11a3 is disposed on the distal-end-side surface 15a in the circumferential direction, and is passed through the fourth hole 15l1.

Concerning the wire 11a passed through the fourth hole 15l1, the wire 11l is extended to the proximal end side of the insertion portion 2.

Also, the second plate body 15 includes, as the plurality of holes, four holes (but not limited to four, but may be an even number of two, or six, or more) for the wire 11b to be passed through. The four holes include a first hole 15d1, a second hole 15d2, a third hole 15r2, and a fourth hole 15r1, in order around the longitudinal axis O (for example, clockwise in FIG. 6).

Concerning the wire 11b, from the proximal end side of the second plate body 15, the wire 11d is passed through the first hole 15d1, is further passed through the second hole 15d2, the third hole 15r2, and the fourth hole 15r1 in order, and the wire 11r is extended to the proximal end side of the second plate body 15.

Similar to the wire 11a shown in FIG. 7, and although illustration of the cross-sectional view is omitted, the wire 11b has the wire 11d passed through the first hole 15d1 from the proximal end side of the insertion portion 2, and is extended as a first part 11b1 of the wire 11b (see FIG. 6) to the distal-end-side surface 15a. The first part 11b1 of the wire 11b is disposed on the distal-end-side surface 15a in the circumferential direction, and is passed through the second hole 15d2.

The wire 11b passed through the second hole 15d2 is extended to the proximal-end-side surface 15b as a second part 11b2 (see FIG. 6). The second part 11b2 is disposed on the proximal-end-side surface 15b in the circumferential direction, and is passed through the third hole 15r2.

The wire 11b passed through the third hole 15r2 is extended to the distal-end-side surface 15a as a third part 11b3 (see FIG. 6). The third part 11b3 is disposed on the distal-end-side surface 15a in the circumferential direction, and is passed through the fourth hole 15r1.

Concerning the wire 11b passed through the fourth hole 15r1, the wire 11r is extended to the proximal end side of the insertion portion 2.

Thus, the wires 11a and 11b are each disposed in the second plate body 15 in an M-shape. Note that the wires 11a and 11b formed in the second plate body 15 in an M-shape may be prepared in advance, and the bending wires 11 may be unitized. In this case, the unitized wires 11 are assembled into the endoscope 1.

As shown in FIG. 8, the proximal-end-side surface 15b of the second plate body 15 is pressed against the guide member 16, which also serves as a bending piece, that is disposed on a distal-most end side. By bringing the wires 11a and 11b into a state of being applied with tension in a tensile direction, the second plate body 15 is fixed to the guide member 16. In this case, neither a work of bonding nor a work of welding the second plate body 15 to the guide member 16 is required, thus enabling to reduce manufacturing costs.

In addition, since the wires 11a and 11b are in the state of being applied with tension, a frictional force acts between the wire 11a and the second plate body 15 and a frictional force acts between the wire 11b and the second plate body 15 at the M-shaped part.

For this reason, pulling the wire 11u does not result in the wire 11l being drawn toward the distal end side, and pulling the wire 11l does not result in the wire 11u being drawn toward the distal end side. Similarly, pulling the wire 11d does not result in the wire 11r being drawn toward the distal end side, and pulling the wire 11r does not result in the wire 11d being drawn toward the distal end side. Thus, the second plate body 15 functions as a wire stop plate.

With the configuration where the single wire 11a serves to function as the up-bending wire 11u and the left-bending wire 11l, and the single wire 11b serves to function as the down-bending wire 11d and the right-bending wire 11r, the need for the work to fix each of distal ends of the four wires to the distal end side of the bending tube 2b (or the proximal end side of the distal end portion 2a) is eliminated, thereby enabling to reduce manufacturing costs. Furthermore, by unitizing the two wires 11a and 11b and the second plate body 15, it is enabled to shorten assembly time and reduce assembly costs.

Note that, although the configuration was described here that uses the two wires 11a and 11b to perform four-directional bending, a general configuration of using individually provided four bending wires to perform four-directional bending can also be adopted for the endoscope 1.

FIG. 9 is a perspective view showing the fixing body 31 disposed in the operation portion 3 in the first embodiment. FIG. 10 is a cross-sectional view showing a portion of a structure of the fixing body 31 in the first embodiment.

The operation portion 3 includes an operation portion body 3a, which also serves as an exterior. The fixing body 31 is fixed to the operation portion body 3a using a screw 32 or the like. The fixing body 31 bundles internal components, such as the treatment instrument channel 21, the gas/liquid feeding channel 22, and a signal cable connected to the image pickup device, and fixes positions of these components in the operation portion 3.

The fixing body 31 includes a receiving surface 31a. The receiving surface 31a is configured, for example, as a surface orthogonal to the longitudinal axis O. At least a portion of the sheath 13 is disposed on the distal end side of the receiving surface 31a.

In the example shown in FIGS. 9 and 10, the entirety of the sheath 13 is disposed on the distal end side of the receiving surface 31a. In this case, a proximal end 13b of the sheath 13 is pressed against the receiving surface 31a. The inner sheath 12 is extended from the proximal end 13b of the sheath 13 to the proximal end side, and a proximal end 12b (see FIG. 9) of the inner sheath 12 is disposed on the proximal end side with respect to the fixing body 31. The proximal end side of the inner sheath 12 is not fixed in the operation portion 3, as mentioned above.

The sheath 13 merely has the one end pressed against the second annular surface 14b of the plate body 14 and the other end pressed against the receiving surface 31a, and is not fixed to any other member.

The sheath 13 is mounted in the endoscope 1, in a compressed state, as mentioned above. For this reason, the proximal end 13b of the sheath 13 pushes the receiving surface 31a of the fixing body 31 by the restoring force of the sheath 13 produced by being compressed.

In other words, the proximal end 13b of the sheath 13 exerts a normal force against the receiving surface 31a. The proximal end 13b of the sheath 13 then produces a frictional force with the receiving surface 31a in a direction intersecting the longitudinal axis O.

At this time, since the receiving surface 31a is orthogonal to the longitudinal axis O, the proximal end 13b of the sheath 13 stably butts against the receiving surface 31a. Note that, if necessary, the receiving surface 31a can be configured to obliquely intersect the longitudinal axis O.

The fixing body 31 further includes a pressing structure portion 31b. The pressing structure portion 31b is provided on the distal end side of the receiving surface 31a of the fixing body 31. The pressing structure portion 31b abuts a side surface of the sheath 13 to prevent buckling of the sheath 13. This prevents buckling of the sheath 13, as well as the inner sheath 12 and the wire 11 that are passed through the sheath 13, thus causing no hindrance to the bending operation with the bending operation knob 6.

A filling rate of the internal components, such as the treatment instrument channel 21, in the inside of the flexible tube 2c is higher than a filling rate in the inside of the operation portion 3. The other internal components then prevent the sheath 13 from shifting in the direction orthogonal to the longitudinal axis O in the flexible tube 2c. For this reason, no structure portion corresponding to the pressing structure portion 31b is provided in the flexible tube 2c. In contrast, the operation portion 3, which has a filling rate of internal components lower than that of the flexible tube 2c, has more space to spare, and therefore is provided with the pressing structure portion 31b.

However, the pressing structure portion 31b of the operation portion 3 can be omitted if the sheath 13, the inner sheath 12, and the wire 11 do not shift in the direction orthogonal to the longitudinal axis O.

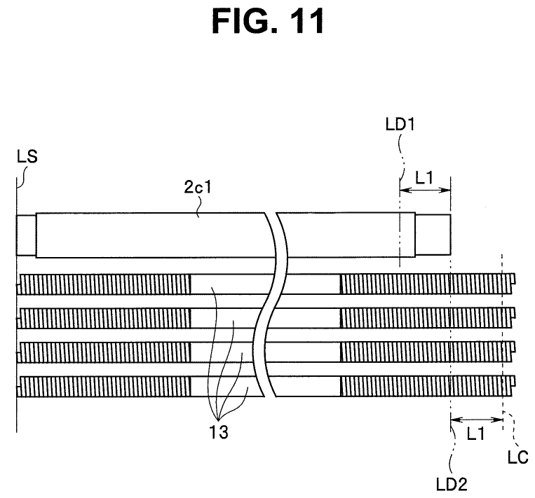

FIG. 11 is a view showing how a position to cut the sheath 13 is set according to an actual length of the flexible tube body 2c1 in the first embodiment.

The actual length of the flexible tube body 2c1 that the flexible tube 2c includes may differ from a design length due to product variation.

The sheath 13 is manufactured by cutting a long coil sheath material. If the actual length of the flexible tube 2c differs from the design length, cutting the sheath 13 at the design length may not result in the sheath 13 in a properly compressed state when mounted in the endoscope 1.

Accordingly, the sheath 13 is cut and formed as shown in FIG. 11 to match the actual length of the flexible tube body 2c1.

In FIG. 11, one end of the flexible tube body 2c1 manufactured is aligned with a start line LS, and a distal end of the coil sheath material is aligned with the start line LS.

The design length of the flexible tube body 2c1 is assumed to be a length from the start line LS to a flexible-tube design line LD1. However, the actual length of the flexible tube body 2c1 in the illustrated example is larger than the length from the start line LS to the flexible-tube design line LD1 by a length L1.

On the other hand, the design length of the sheath 13 is assumed to be a length from the start line LS to a sheath design line LD2. In this case, corresponding to the fact that the flexible tube body 2c1 is longer than the design length by the length L1, a cutting line LC to cut the sheath 13 is set at a position longer than the sheath design line LD2 by the length L1, to cut the long coil sheath material.

By adopting such a manufacturing method, it is enabled that the sheath 13 maintains a properly compressed state when mounted in the endoscope 1, even when there is product variation in the length of the flexible tube body 2c1.

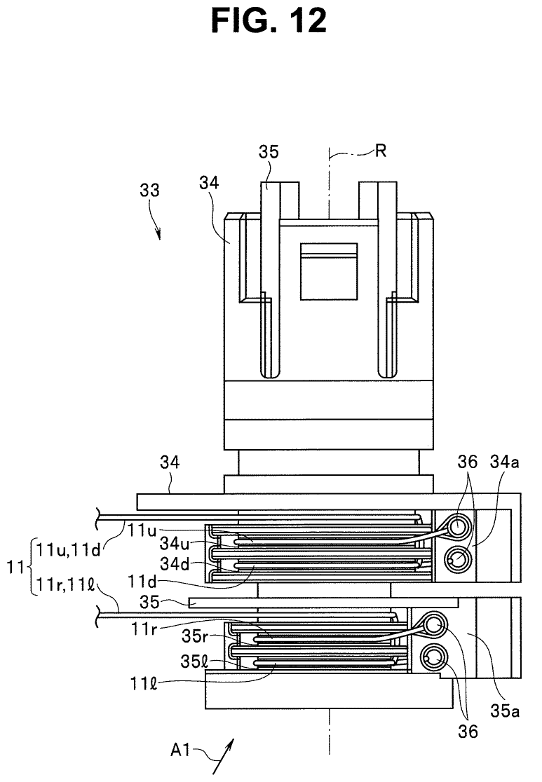

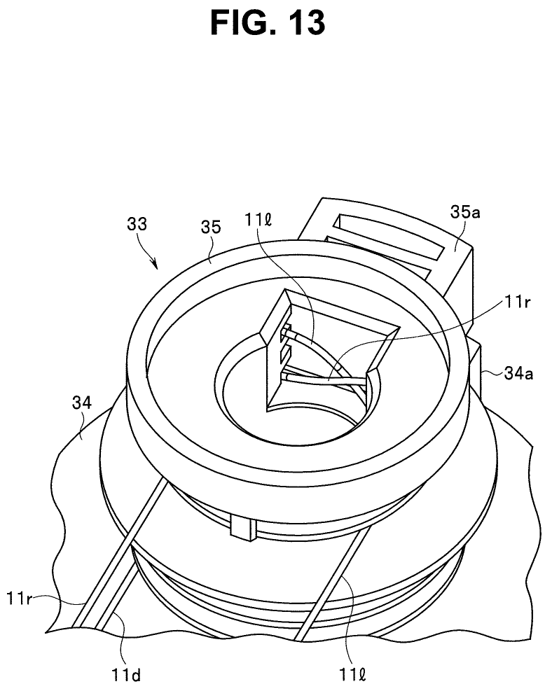

FIG. 12 is a side view of the operation mechanism 33 disposed in the operation portion 3 in the first embodiment. FIG. 13 is a partial perspective view showing the operation mechanism 33 of the first embodiment from a direction of an arrow A1 in FIG. 12.

The operation portion 3 includes the operation mechanism 33 configured to receive an operation to pull or loosen the wire 11. The proximal end side of the wire 11 is fixed to the operation mechanism 33.

The operation mechanism 33 includes a pulley 34 and a pulley 35. The pulley 34 pivots around a rotation axis R in conjunction with a pivoting operation of the up-down bending operation knob 6a. The pulley 35 pivots around the rotation axis R in conjunction with a pivoting operation of the left-right bending operation knob 6b.

Note that, although an example has been shown here where the operation mechanism 33 is in conjunction with the bending operation knob 6, the operation mechanism 33 is not limited to this configuration. For example, the operation mechanism 33 can be configured to be electrically pivoted by an actuator or the like in response to a joystick operation, or may adopt any other appropriate configuration.

Around the pulley 34, the up-bending wire 11u and the down-bending wire 11d are wound, and to the pulley 34, the proximal end side of the wire 11u and the proximal end side of the wire 11d are fixed. Around the pulley 35, the right-bending wire 11r and the left-bending wire 11l are wound, and to the pulley 35, the proximal end side of the wire 11r and the proximal end side of the wire 11l are fixed.

The pulley 34 includes an up-bending wire winding chamber 34u and a down-bending wire winding chamber 34d, which are located at different positions in the direction of the rotation axis R. The up-bending wire winding chamber 34u and the down-bending wire winding chamber 34d are separated from each other by, for example, a flange or the like interposed therebetween.

The pulley 35 includes a right-bending wire winding chamber 35r and a left-bending wire winding chamber 35l, which are located at different positions in the direction of the rotation axis R. The right-bending wire winding chamber 35r and the left-bending wire winding chamber 35l are separated from each other by, for example, a flange or the like interposed therebetween.

For example, the wire 11r and the wire 11l are crossed on the proximal end side of pulley 35, as shown in FIG. 13. The wire 11r is crossed, is then wound once around the right-bending wire winding chamber 35r, and is fixed to a fixation portion 35a of the pulley 35 by a first knock pin 36. The wire 11l is crossed, is then wound once around the left-bending wire winding chamber 35l, and is fixed to the fixation portion 35a of the pulley 35 by a second knock pin 36. With such a configuration, the wires 11r and 11l do not overlap, and each can transmit a pulling force and each can loosen.

The wires 11u and 11d are fixed to the pulley 34 in a similar manner as the wires 11r and 11l. That is, the wires 11u and 11d are crossed on the proximal end side of the pulley 34. The wire 11u is crossed, is the wound once around the up-bending wire winding chamber 34u, and is fixed to a fixation portion 34a of the pulley 34 by a third knock pin 36. The wire 11d is crossed, is then wound once around the down-bending wire winding chamber 34d, and is fixed to the fixation portion 34a of the pulley 34 by a fourth knock pin 36. With such a configuration, the wires 11u and 11d do not overlap, and each can transmit a pulling force and each can loosen.

Here, standard general-purpose parts may be used as the first to fourth knock pins 36. By using general-purpose parts, the manufacturing cost of endoscope 1 can be reduced compared to a case of using dedicated parts.

As described with reference to FIGS. 6 to 8, when the wire 11u and the wire 11l are configured of the single wire 11a, the wire 11a has the one end (the proximal end of the wire 11u) fixed to the pulley 34, is extended to the distal end side, is passed through the flexible tube 2c and the bending tube 2b, is disposed in the second plate body 15 in an M-shape to be extended to the proximal end side, is passed through the bending tube 2b and the flexible tube 2c, and has the other end (the proximal end of the wire 11l) fixed to the pulley 35.

Similarly, when the wire 11d and the wire 11r are configured of the single wire 11b, the wire 11b has the one end (the proximal end of the wire 11d) fixed to the pulley 34, is extended to the distal end side, is passed through the flexible tube 2c and the bending tube 2b, is disposed in the second plate body 15 in an M-shape to be extended to the proximal end side, is passed through the bending tube 2b and the flexible tube 2c, and has the other end (the proximal end of the wire 11r) fixed to the pulley 35.

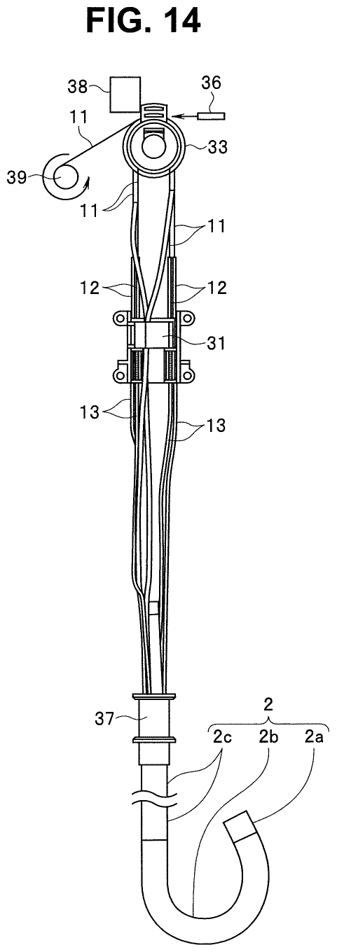

FIG. 14 is a view for illustrating a process of fixing the bending wire 11 to the operation mechanism 33 in the first embodiment.

As mentioned above, the wire 11 is fixed in the endoscope 1 in a state of being applied with tension. The fixing of the wire 11 inside the endoscope 1 is performed, for example, by the following process.

A bending tube base 37, the fixing body 31, and the operation mechanism 33 that are provided on the distal end side of the operation portion 3 are each fixed to a jig at positions separated by respective specified distances from each other.

Furthermore, the fixation portions 34a and 35a of the operation mechanism 33 are pressed against a rotation-stopping jig 38 to hold the pulleys 34 and 35 in a non-rotating state.

The proximal end side of the wire 11 is then wound around a winding jig 39, and the winding jig 39 is rotated to wind the proximal end side of the wire 11.

When the wire 11 is wound by the jig 39, the bending tube 2b bends in accordance with the winding amount. Once the bending tube 2b is in a maximum bending state, the winding force of the jig 39 is adjusted to obtain a state where the wire 11 is tensioned at a constant tension.

Once this state is obtained, the knock pin 36 is press-fitted into each of the fixation portions 34a and 35a to fix the proximal end side of the wire 11.

By doing such a work for each of the wires 11u, 11d, 11r, and 11l, a state can be obtained in which a constant tension is applied to each of the wires 11u, 11d, 11r, and 11l even when the bending tube 2b is in a straight-line state.

Here, if hypothetically the wire 11 is not applied with a constant tension, it exhibits slack. If this is the case, even if the bending operation starts, the bending will not start until the wire 11 is taut, thus reducing the responsiveness of the bending to the user operation.

In contrast, the wire 11 fixed in the above-mentioned process is in a state of being applied with a constant tension, thus eliminating slack and enabling bending to be performed with high responsiveness to user operation.

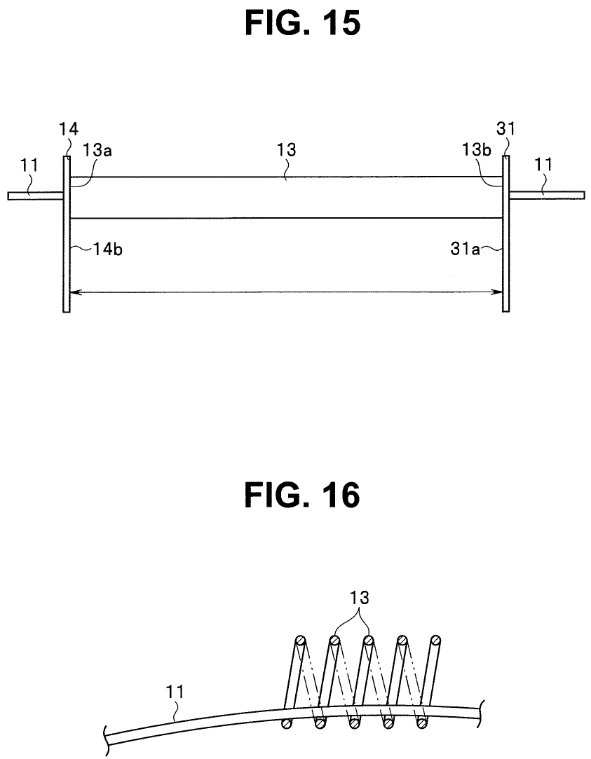

FIG. 15 is a view showing that the sheath 13 is held in a state of pressing the plate body 14 and the fixing body 31 in the first embodiment.

The sheath 13 has the distal end 13a pressed against the second annular surface 14b of the plate body 14, and the proximal end 13b pressed against the receiving surface 31a of the fixing body 31, thereby pushing the plate body 14 and the fixing body 31, as shown by both arrows.

As a configuration that operates in this manner, an example was mentioned above in which the sheath 13 is a coil sheath in a compressed state. However, the sheath 13 is not limited to a coil sheath and can have any other configuration having elasticity. For example, the sheath 13 can be a tubular member formed of elastic rubber.

Furthermore, the sheath 13 does not have to be configured of a common elastic material (a material having compressibility). For example, a small-diameter endoscope may adopt a thin-walled metal pipe as a tubular member that configures a channel. Even a metal pipe, if capable of being mounted in the endoscope 1 in a state where both ends of the metal pipe are pressed, will induce a normal force against the plate body 14 and the fixing body 31, and produce a frictional force in a direction intersecting the longitudinal axis O between the plate body 14 and the fixing body 31. Therefore, the sheath 13 may be a cylindrical member, such as a metal pipe, formed of a rigid material.

FIG. 16 is a cross-sectional view showing a relationship between the sheath 13 and the wire 11 when the inner sheath 12 is not provided in the first embodiment.

The wire 11 moves in the direction of the longitudinal axis O when pulled or loosened. On the other hand, the sheath 13 is sandwiched between the plate body 14 and the fixing body 31, as shown in FIG. 15, and does not move in the direction of the longitudinal axis O. Therefore, when the wire 11 is pulled or loosened, a relative position change in the direction of the longitudinal axis O will occur between the wire 11 and the sheath 13.

If hypothetically the inner sheath 12 is not provided, as shown in FIG. 16, when the positions of the wire 11 and the sheath 13 change relative to each other, the wire 11 contacts and causes friction on the surface of the core wire of the sheath 13, which is configured as a coil sheath, and the wire 11 may wear. Furthermore, when the positions of the wire 11 and the sheath 13 change relative to each other, the wire 11 contacts and causes friction on an edge of an end portion of the sheath 13, and the wire 11 may wear.

In contrast, in the present embodiment, as mentioned above, the inner sheath 12 has the distal end 12a disposed on the distal end side with respect to the hole 14c of the plate body 14, and has the proximal end 12b disposed on the proximal end side with respect to the proximal end 13b of the sheath 13.

In other words, the inner sheath 12 is passed through and over the entire length of the sheath 13, ensuring that the wire 11 does not come into direct contact with the sheath 13. Furthermore, as mentioned above, the friction coefficient between the inner sheath 12 and the wire 11 is less than the friction coefficient between the sheath 13 and the wire 11.

This prevents the wire 11 from contacting the surface or edge of the core wire of the sheath 13 and wearing. Furthermore, an increase in the amount of force due to the friction can be suppressed when the wire 11 is pulled.

In addition, the distal end 12a of the inner sheath 12 is disposed on the distal end side with respect to the hole 14c. The wire 11 then not only does not contact the sheath 13, but also does not contact the plate body 14. This enables to prevent the wire 11 from contacting the surface or edge of the plate body 14 and wearing.

The proximal end 12b of the inner sheath 12 is disposed on the proximal end side with respect to the fixing body 31, as mentioned above. The wire 11 then does not contact the fixing body 31 either. This enables to prevent the wire 11 from contacting the surface or edge of the fixing body 31 and wearing.

According to the first embodiment, the sheath 13 is not fixed, only having the one end pushing the plate body 14 and the other end pushing the fixing body 31. Therefore, there is no need for skilled workers to use a jig to conduct the welding work and bonding work when mounting the sheath 13 in the endoscope 1. For this reason, the endoscope 1, which includes the sheath 13 through which the bending wire 11 is passed, is suitable for inexpensive manufacturing.

Furthermore, since the plate body 14 is fixed by the pressing force of the sheath 13, and the second plate body 15 is fixed by the tension of the wire 11, welding and bonding are unnecessary for both the plate body 14 and the second plate body 15. Thus, it is enabled to manufacture the endoscope 1 more inexpensively.

In addition, by differentiating the position of the hole 14c in the plate body 14 and the position of the hole 16a in the guide member 16, the wire 11 is given a swing angle. For this reason, it is enabled to dispose the wire 11 in different manners in the flexible tube 2c and the bending tube 2b, thereby increasing the degree of freedom in the disposition of the internal components.

### SECOND EMBODIMENT

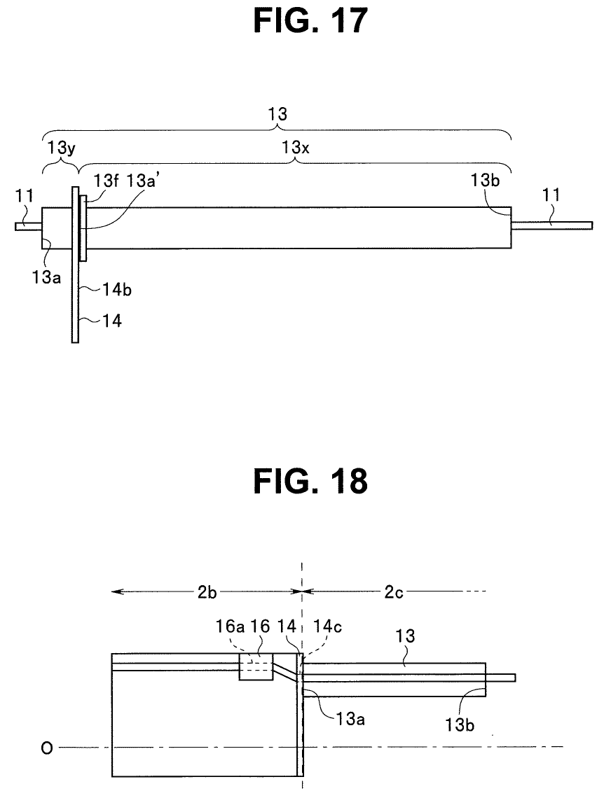

FIG. 17 is a view showing a configuration example of the sheath 13 in a second embodiment of the present disclosure. In the second embodiment, parts similar to those in the first embodiment are attached with the same symbols, and descriptions thereof are omitted as appropriate. In the second embodiment, points different from the first embodiment are mainly described.

The sheath 13 has a portion 13x disposed on the proximal end side of the second annular surface 14b of the plate body 14, and has another portion 13y disposed on the distal end side of the second annular surface 14b of the plate body 14. The portion 13x is longer than the other portion 13y in the direction of the longitudinal axis O.

A flange 13f protruding radially outward is provided at a distal end of the portion 13x of the sheath 13. Therefore, the position where the flange 13f is provided is in the distal end side of the sheath 13, but not at the distal end 13a of the entire sheath 13.

The flange 13f of the sheath 13 abuts the second annular surface 14b to push the plate body 14. The surface of the flange 13f, which abuts the second annular surface 14b, is a distal end 13a′ of the portion 13x of the sheath 13. Therefore, the distal end 13a′ of the portion 13x of the sheath 13 is the section that pushes the plate body 14.

Note that a configuration can be employed to provide, instead of the flange 13f, a projection or the like protruding radially outward from the sheath 13, to push the plate body 14 by the projection or the like.

If the sheath 13 is formed of a non-compressible material, a configuration may be employed to provide a separate elastic member, so that the elastic member presses the flange 13f against the plate body 14.

Furthermore, instead of the configuration to use the flange 13f to push the plate body 14, a configuration is also employable to bond a portion of an outer circumferential surface of the sheath 13 integrally with the plate body 14, so that the sheath 13 presses the plate body 14.

According to the second embodiment, an effect almost the same as that of the above-mentioned first embodiment is yielded, even when a part that is not the distal end 13a of the entire sheath 13, i.e., the distal end 13a′ of the portion 13x of the sheath 13, which is disposed on the proximal end side of the plate body 14, is configured to push the plate body 14.

### THIRD EMBODIMENT

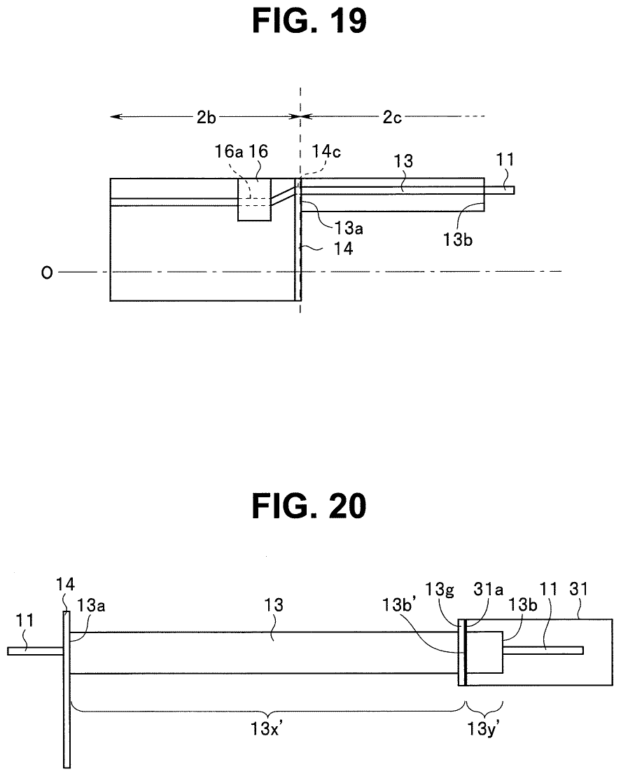

FIGS. 18 and 19 show a third embodiment of the present disclosure. In the third embodiment, parts similar to those in the first and second embodiments are attached with the same symbols, and descriptions a thereof are omitted as appropriate. In the third embodiment, points different from the first and second embodiments are mainly described.

As mentioned above, by differentiating the position of the hole 14c in the plate body 14 and the position of the hole 16a in the guide member 16, the wire 11 can be given a swing angle.

FIG. 18 is a view showing a first example of the swing angle of the wire 11 in the third embodiment.

In the plane orthogonal to the longitudinal axis O, the hole 14c in the plate body 14 is provided at a position closer to the longitudinal axis O than the position where the guide member 16 holds the wire 11, i.e., than the hole 16a in the guide member 16. The swing angle of the first example shown in FIG. 18 is almost the same as the swing angle of the wire 11 described in the first embodiment.

In this case, the swing angle of the wire 11 is a swing angle to a radially outward side as the wire 11 extends from the flexible tube 2c side toward the bending tube 2b side. By swinging the wire 11 to a radially outward side to widen the internal space of the bending tube 2b, it is enabled to increase the degree of freedom in the disposition of the internal components in the bending tube 2b.

FIG. 19 is a view showing a second example of the swing angle of the wire 11 in the third embodiment.

In the plane orthogonal to the longitudinal axis O, the hole 14c in the plate body 14 is provided at a position farther from the longitudinal axis O than the position where the guide member 16 holds the wire 11, i.e., than the hole 16a in the guide member 16.

In this case, the swing angle of the wire 11 is a swing angle to a radially inward side as the wire 11 extends from the flexible tube 2c side to the bending tube 2b side. The bending tube 2b tends to have a thicker outer skin and therefore a larger outer diameter. For this reason, by swinging the wire 11 to a radially inward side to narrow the inside of the bending tube 2b, it is enabled to suppress the outer diameter of the bending tube 2b from becoming larger.

Note that the above showed an example of shifting the positions of the hole 14c and the hole 16a in the radial direction from each other, but no limitation is placed thereon and positions of the hole 14c and the hole 16a in the circumferential direction can be shifted from each other as well. In this case, it is facilitated to adjust the disposition of the internal components.

According to the third embodiment, an effect almost the same as that of the above-mentioned first and second embodiments is yielded.

According to the third embodiment, by providing a swing angle to the wire 11, it is enabled to increase the degree of freedom in the disposition of the internal components, facilitate the adjustment of the disposition of the internal components, or suppress the outer diameter of the bending tube 2b from becoming larger.

### FOURTH EMBODIMENT

FIG. 20 shows a fourth embodiment of the present disclosure. In the fourth embodiment, parts similar to those in the first to third embodiments are attached with the same symbols, and descriptions thereof are omitted as appropriate. In the fourth embodiment, points different from the first to third embodiments are mainly described.

FIG. 20 is a view showing a configuration example of the sheath 13 in a fourth embodiment.

The sheath 13 has a portion 13x′ disposed on the distal end side of the receiving surface 31a of the fixing body 31, and has another portion 13y′ disposed on the proximal end side of the receiving surface 31a of the fixing body 31. The portion 13x′ is longer than the other portion 13y′ in the direction of the longitudinal axis O.

A flange 13g protruding radially outward is provided at a proximal end of the portion 13x′ of the sheath 13. Therefore, the position where the flange 13g is provided is on the proximal end side of the sheath 13, but not at the proximal end 13b of the entire sheath 13.

The flange 13g of the sheath 13 abuts the receiving surface 31a of the fixing body 31 to push the fixing body 31. The surface of the flange 13g, which abuts the receiving surface 31a, is the proximal end 13b′ of the portion 13x′ of the sheath 13. Therefore, the proximal end 13b′ of the portion 13x′ of the sheath 13 is the section that pushes the fixing body 31.

Note that a configuration can be employed to provide, instead of the flange 13g, a projection or the like protruding radially outward from the sheath 13, to push the fixing body 31 by the projection or the like.

If the sheath 13 is formed of a non-compressible material, a configuration may be employed to provide a separate elastic member, so that the elastic member presses the flange 13g against the fixing body 31.

Furthermore, the sheath 13 can be configured to include both the flange 13f shown in FIG. 17 and the flange 13g shown in FIG. 20.

According to the fourth embodiment, an effect almost the same as that of the above-mentioned first to third embodiments is yielded, even when a part that is not the proximal end 13b of the entire sheath 13, i.e., the proximal end 13b′ of the portion 13x of the sheath 13, which is disposed on the distal end side of the fixing body 31, is configured to push the fixing body 31.

### FIFTH EMBODIMENT

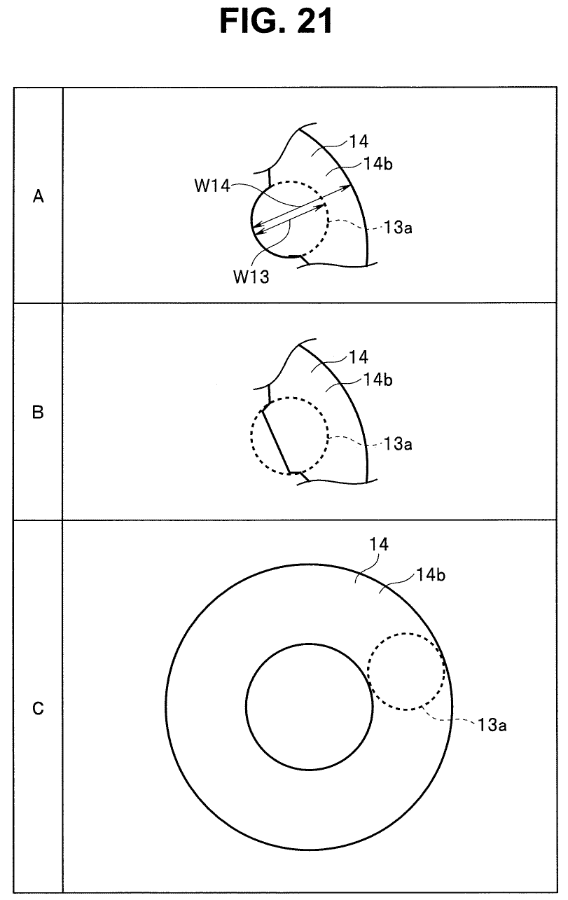

FIG. 21 shows a fifth embodiment of the present disclosure. In the fifth embodiment, parts similar to those in the first to fourth embodiments are attached with the same symbols, and descriptions thereof are omitted as appropriate. In the fifth embodiment, points different from the first to fourth embodiments are mainly described.

FIG. 21 is a diagram showing a configuration example of a part of the second annular surface 14b of the plate body 14, where the distal end 13a of the sheath 13 abuts, in a fifth embodiment.

As mentioned above, the plate body 14 is formed as a ring member for the internal components to be passed through. In this case, a ring width of the plate body 14 in the plane orthogonal to the longitudinal axis O should be small so as not to interfere with the passing through of the internal components.

However, the plate body 14 receives a force pushed from the distal end 13a of the sheath 13. For this reason, the width W14 of the part of the plate body 14, where the distal end 13a abuts, can be greater than or equal to the width (outer diameter) W13 of the distal end 13a of the sheath 13. In the example shown in column A of

FIG. 21, the plate body 14 is configured such that W14≥W13 (in the illustrated exemplary example, W14>W13). However, the width of the plate body 14 other than the part where the distal end 13a abuts is less than W14. Therefore, the part of the plate body 14, which has the width W14, is shaped to protrude radially inward.

According to the configuration example shown in column A of FIG. 21, both improved passability of the internal components and reception of a stable pressing force by the plate body 14 from the distal end 13a of the sheath 13 can be achieved.

An example shown in column B of FIG. 21 is one in which the width of the part of the plate body 14, where the distal end 13a abuts, is smaller than that in column A of FIG. 21. In this case, the entirety of the distal end 13a does not abut the second annular surface 14b of the plate body 14, but only a portion of the distal end 13a abuts the second annular surface 14b.

In the configuration shown in column A of FIG. 21, the distal end 13a is pressed against the second annular surface 14b in a circular shape, while in the configuration shown in column B of FIG. 21, the distal end 13a is pressed against the second annular surface 14b in a D shape (shape of a portion of a circular arc). If the plate body 14 can receive a stable pressing force from the distal end 13a of the sheath 13, the configuration shown in column B of FIG. 21 can be adopted.

According to the configuration example shown in column B of FIG. 21, the filling rate of the internal components that are passed through the plate body 14 can be decreased. Furthermore, the materials used to manufacture the plate body 14 can be reduced.

On the other hand, the example shown in column C of FIG. 21 is an example in which the width of the plate body 14 in the plane orthogonal to the longitudinal axis O is a constant width greater than or equal to the width of the distal end 13a of the sheath 13. If there is enough space in the flexible tube 2c with no hindrance for passage through of the internal components, the configuration shown in column C of FIG. 21 can be adopted. According to the configuration example shown in column C of FIG. 21, the aligning work to the part having a wide ring width is unnecessary when providing four holes 14c in the plate body 14.

According to the fifth embodiment, an effect almost the same as that of the above-mentioned first to four embodiments is yielded.

According to the fifth embodiment, it is enabled to select the plate body 14 with an appropriate configuration according to the stability of pressing the sheath 13 against the plate body 14, the need to decrease the filling rate of the internal components, the ease of the work to provide the hole 14c in the plate body 14, etc.

Note that the present disclosure is not limited to the above-mentioned embodiments as-is. The present disclosure can be embodied by modifying the components to an extent not departing from the gist of the present disclosure, at the implementation stage. Also, a plurality of components disclosed in the above-mentioned embodiments can be combined as appropriate to form various aspects of the present disclosure. For example, some components may be deleted from all the components disclosed in the embodiments. Furthermore, components of different embodiments may be combined as appropriate. Thus, it is of course possible to make various variations and applications within the scope not departing from the essence of the present disclosure.

What is claimed is:

1. An endoscope comprising:
a bending tube extending along a longitudinal axis from a proximal end side to a distal end side;
a wire configured to bend the bending tube;
a first sheath through which the wire is passed; and
a plate body having an annular shape and disposed in the proximal end side of the bending tube, wherein
the plate body comprising:
a first annular surface facing distally;
a second annular surface facing proximally and intersecting the longitudinal axis; and
a hole penetrating the first annular surface and the second annular surface, and through which the wire is passed, and
at least a portion of the first sheath is disposed proximally relative to the second annular surface, and a distal end of at least the portion of the first sheath is configured to push a periphery of the hole on the second annular surface.

2. The endoscope according to claim 1, wherein the first sheath has elasticity, and the distal end of at least the portion of the first sheath is configured to push the periphery of the hole on the second annular surface, by a restoring force produced by the first sheath compressed in a direction of the longitudinal axis.

3. The endoscope according to claim 2, wherein the first sheath is a coil sheath.

4. The endoscope according to claim 1, further comprising:
a second sheath passed through the first sheath, and through which the wire is passed, wherein
the second sheath is passed through the hole, and a distal end of the second sheath extends distally relative to the hole.

5. The endoscope according to claim 4, wherein a portion of the second sheath is bonded to the hole.

6. The endoscope according to claim 4, wherein a first friction coefficient between the second sheath and the wire is less than a second friction coefficient between the first sheath and the wire.

7. The endoscope according to claim 6, wherein
the second sheath is formed of resin, and
the second annular surface extends orthogonal to the longitudinal axis.

8. The endoscope according to claim 1, wherein an entirety of the first sheath is disposed proximally relative to the plate body, and a distal end of the first sheath pushes the periphery of the hole on the second annular surface.

9. The endoscope according to claim 8, wherein
a width of the periphery of the hole on the second annular surface, is greater than or equal to a width of the distal end of the first sheath, and
an entire surface of the distal end of the first sheath abuts the second annular surface.

10. The endoscope according to claim 1, wherein
the bending tube includes a guide member that holds the wire, and
the hole is provided at a first radial position different from a second radial position where the guide member holds the wire.

11. The endoscope according to claim 10, wherein the first radial position is closer to the longitudinal axis than the second radial position.

12. The endoscope according to claim 10, wherein the first radial position is farther from the longitudinal axis than the second radial position.

13. The endoscope according to claim 1, further comprising:
a flexible tube provided proximally relative to the bending tube; and
an operation portion provided proximally relative to the flexible tube, wherein
the operation portion includes a fixing body including a receiving surface orthogonal to the longitudinal axis, and
at least the portion of the first sheath is disposed distally relative to the receiving surface, and a proximal end of at least the portion is configured to push the receiving surface.

14. The endoscope according to claim 13, wherein an entirety of the first sheath is disposed distally relative to the receiving surface, and a proximal end of the first sheath is configured to push the receiving surface.

15. The endoscope according to claim 13, wherein the fixing body includes a pressing structure arranged distally relative to the receiving surface, the pressing structure configured to abut a side surface of the first sheath to prevent buckling of the first sheath.

16. The endoscope according to claim 13, wherein
the operation portion includes an operation mechanism configured to receive an operation to pull or loosen the wire, and
a proximal end side of the wire is fixed to the operation mechanism.

17. The endoscope according to claim 16, wherein
the operation mechanism includes a pulley around which the wire is wound, and
the proximal end side of the wire is fixed to the pulley by a knock pin.

18. The endoscope according to claim 1, further comprising:
a second plate body disposed on the distal end side of the bending tube, wherein
the second plate body includes a first hole, a second hole, a third hole, and a fourth hole in order around the longitudinal axis, the first to fourth holes penetrating a distal-end-side surface and a proximal-end-side surface of the second plate body, and
the wire is passed, in order, through the first hole from a proximal end side of the second plate body, through the second hole from a distal end side of the second plate body, through the third hole from the proximal end side of the second plate body, and through the fourth hole from the distal end side of the second plate body.

19. An endoscope comprising:
a bending tube extending along a longitudinal axis from a proximal end side to a distal end side;
a wire configured to bend the bending tube;
a first sheath through which the wire is passed; and
a plate body having an annular shape and disposed in the proximal end side of the bending tube, wherein
the plate body comprising:
a first annular surface facing distally;
a second annular surface facing proximally and intersecting the longitudinal axis; and
a hole penetrating the first annular surface and the second annular surface, and through which the wire is passed, and
at least a portion of the first sheath is disposed proximally relative to second annular surface, and a distal end of at least the portion of the first sheath is configured to produce a frictional force in a direction intersecting the longitudinal axis between the distal end of at least the portion and a periphery of the hole on the second annular surface.

20. An endoscope comprising:
a bending tube extending along a longitudinal axis from a proximal end side to a distal end side;
a wire configured to bend the bending tube;
a first sheath;
a second sheath passed through the first sheath, and through which the wire is passed; and
a plate body having an annular shape and disposed in the proximal end side of the bending tube, wherein
the plate body includes:
a first annular surface facing distally;
a second annular surface facing proximally; and
a hole penetrating the first annular surface and the second annular surface, and through which the wire is passed,
at least a portion of the first sheath is disposed proximally relative to the second annular surface, and a distal end of at least portion of the first sheath is configured to push a periphery of the hole on the second annular surface, and
the wire and the second sheath are passed through the hole, and a distal end of the second sheath is disposed distally relative to the hole.
